(19) United States
(12) Patent Application Publication
(10) Pub. No.: US 2011/0070195 A1
(43) Pub. Date: Mar. 24, 2011

(54) USES OF SPATIAL CONFIGURATION TO MODULATE PROTEIN FUNCTION

(76) Inventor: Guangwen Wei, Sichuan (CN)

(21) Appl. No.: 12/889,521

(22) Filed: Sep. 24, 2010

Related U.S. Application Data
(63) Continuation of application No. 10/927,975, filed on Aug. 26, 2004, now abandoned.
Application No. 10/927,975 is a continuation of application No. 10/650,365, filed on Aug. 28, 2003, now Pat. No. 7,364,724.
Application No. 10/650,365 is a continuation-in-part of application No. PCT/CN02/00128, filed on Feb. 28, 2002.
(60) Provisional application No. 60/498,449, filed on Aug. 28, 2003.
(60) Provisional application No. 60/498,785, filed on Aug. 28, 2003.
(60) Provisional application No. 60/498,923, filed on Aug. 28, 2003.

(30) Foreign Application Priority Data
Feb. 28, 2001 (CN) 01104367.9
Mar. 5, 2004 (IN) 279/MUM/2004
Mar. 5, 2004 (IN) 280/MUM/2004

(51) Int. Cl. A61K 38/21; C07K 14/555; C07H 21/00; C12N 15/63; C12P 21/00; C12Q 1/68; C12N 1/21; A61P 31/12

(52) U.S. Cl. 424/85.4; 530/351; 536/23.5; 435/320.1; 435/69.51; 435/6; 435/252.33

(57) ABSTRACT

This invention provides a set of methods for modulating protein spatial configuration. First, select the amino-acid codon for encoding the target protein according to host codon usage. Second, choose combinations which can modulate the spatial configuration and construct into different vectors which can transfect a series of hosts. Third, choose the vector promoter by monitoring a combination of base pairs after combining the code sequence of the promoter and the target protein. Finally, choose the appropriate expression host to express the target protein, refold and purify, measure the activity and spatial configuration.

The application disclosed herein claims benefit of U.S. Ser. No. 60/498,449, filed Aug. 28, 2003; U.S. Ser. No. 60/498,785, filed Aug. 28, 2003; U.S. Ser. No. 60/498,923, filed Aug. 28, 2003; and U.S. Ser. No. 10/650,365, filed Aug. 28, 2003, which is a continuation-in-part of Int'l App'l No. PCT/CN02/00128, filed Feb. 28, 2002, which claims priority of Chinese Application No. 01104367.9, filed Feb. 28, 2001. This application claims priority of Indian Application No. 279/MUM/2004, filed Mar. 5, 2004, and Indian Application No. 280/MUM/2004, filed Mar. 5, 2004. The contents of the preceding applications are hereby incorporated in their entireties by reference into this application.

Throughout this application, various publications are referenced. Disclosures of these publications in their entireties are hereby incorporated by reference into this application in order to more fully describe the state of the art to which this invention pertains.

### BACKGROUND OF THE INVENTION

The completion of the human genome project verified the therapeutic effects of many genes, and some of them have been developed into therapeutic proteins, but most of them cannot be controlled by gene or protein techniques in the art. They cannot be correctly translated into proteins which maintain the whole therapeutic effects possessed by their genes. The biggest obstacle on the road to successful protein translation is the correct protein-folding. The field of research on how to obtain a protein with efficient spatial configuration is filled with competition.

Changing the spatial configuration of proteins without disturbing amino acid sequence may change functions of certain proteins. For example, some proteins with abnormal 3-dimensional structure can cause diseases in humans and animals, such as: bovine spongiform encephalopathy (BSE), Alzheimer's Disease, cystic fibrosis, familial hypercholestrolacemia, familial amyloid disease, certain carcinoma or cataract. These diseases also have been called “folding-diseases”. The “Prion” protein causes BSE and can infect normal proteins and transmit among them.

During the research of protein structure, most researchers consider that the most important part in retrieving the correct spatial structure of proteins are the techniques of denaturation and refolding. Masses of literature reported improvement in refolding associated with various chaperons or reverse micelles, etc. Many secretion expression vectors have been developed to allow those proteins expressed in more natural environments, but all these efforts only result in an increase in the yields of proteins, not in qualitative changes.

### DETAILED DESCRIPTION OF THE FIGURES

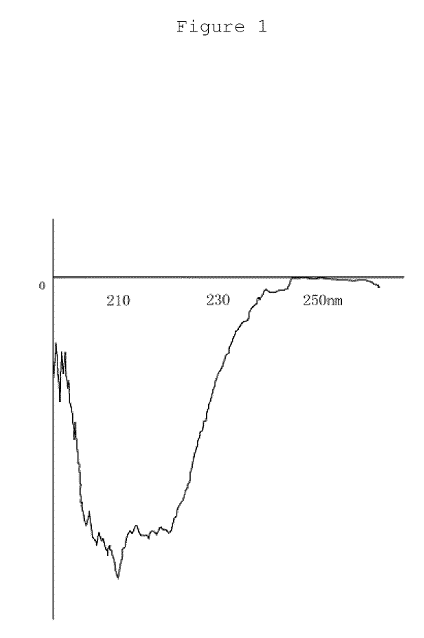

FIG. 1. Circular Dichroism spectrum of Infergen®

Spectrum range: 250 nm-190 nm

Sensitivity: 2 m°/cm

Light path: 0.20 cm

Equipment: Circular Dichroism J-500C

Samples: contain 30 μg/ml IFN-con1, 5.9 mg/ml of NaCl and 3.8 mg/ml of Na₂PO₄, pH7.0.

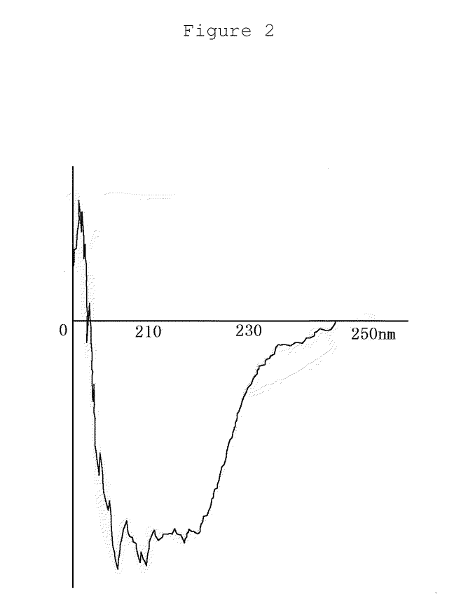

FIG. 2. Circular Dichroism spectrum of rSIFN-co

Spectrum range: 250 nm-190 nm

Sensitivity: 2 m°/cm

Light path: 0.20 cm

### Equipment: Circular Dichroism J-500C

Samples: contain 30 μg/ml rSIFN-co, 5.9 mg/ml of NaCl and 3.8 mg/ml of Na₂PO₄, pH7.0.

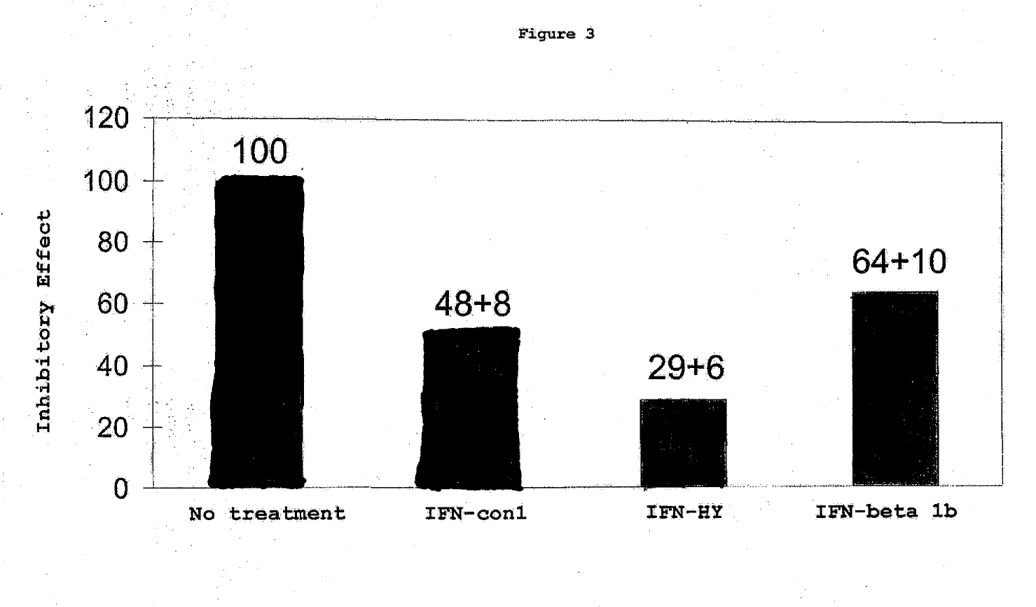

FIG. 3. Comparison of Inhibition Effects of Different Interferons on HBV Gene Expression

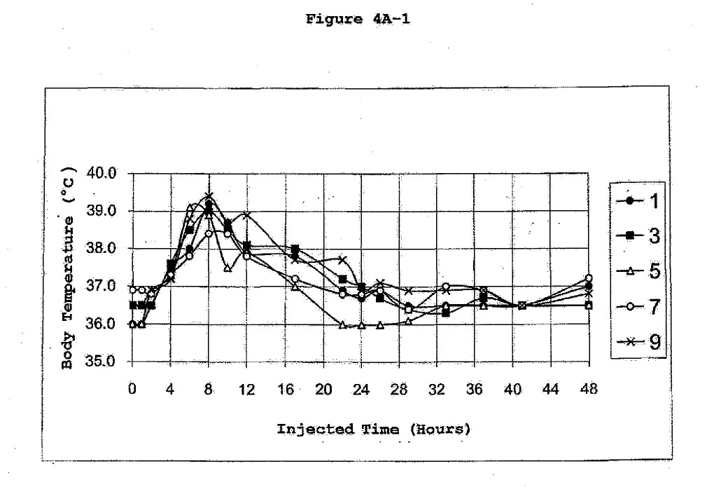

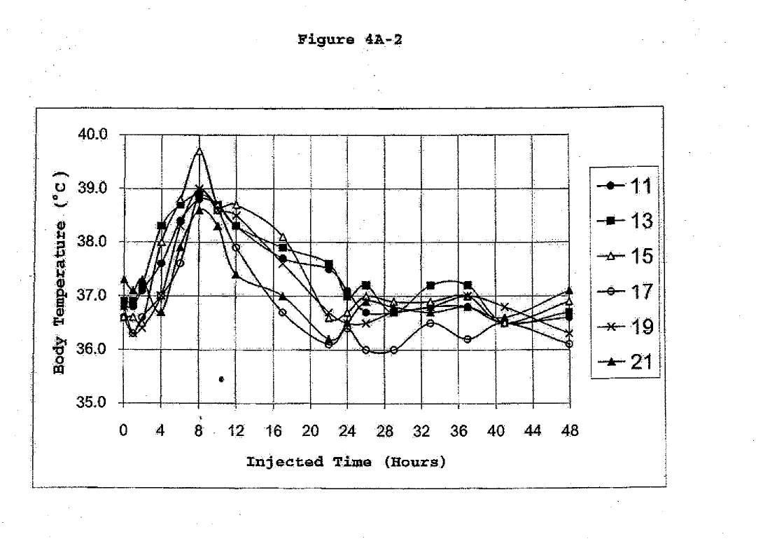

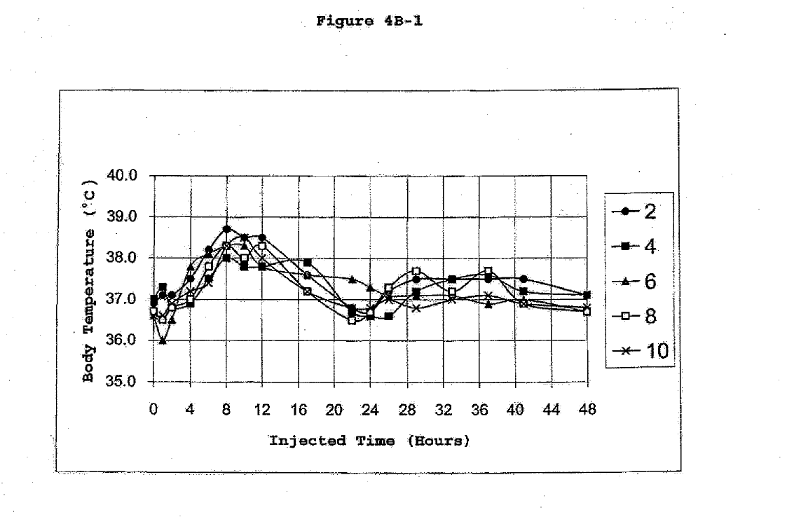

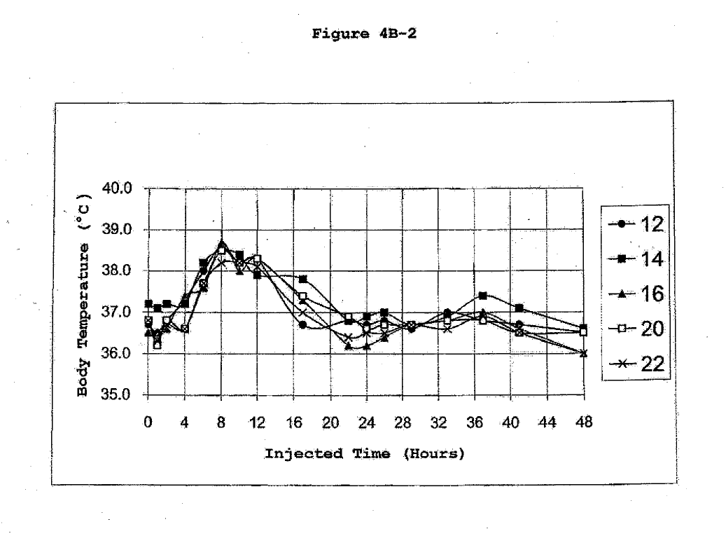

FIG. 4A-1. Curves of Changes of Body Temperature in Group A (5 patients)

This figure is the record of body temperature changes of 5 patients in Group A.

FIG. 4A-2. Curves of Changes of Body Temperature in Group A (6 patients)

This figure is the record of body temperature changes of the other 6 patients in Group A.

FIG. 4B-1. Curves of Changes of Body Temperature in Group B (5 patients)

This figure is the record of body temperature changes of 5 patients in Group B.

FIG. 4B-2. Curves of Changes of Body Temperature in Group B (5 patients)

This figure is the record of body temperature changes of the other 5 patients in Group B.

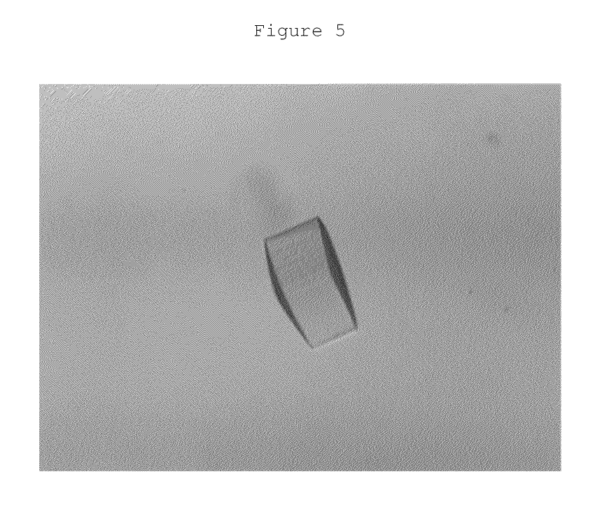

FIG. 5. rsIFN-co Crystal I

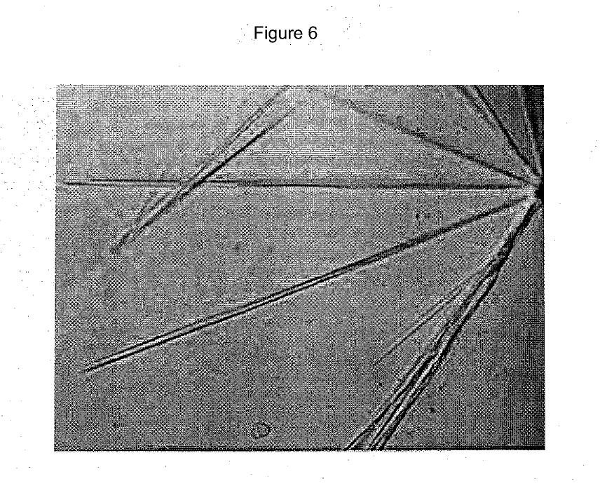

FIG. 6. rsIFN-co Crystal II

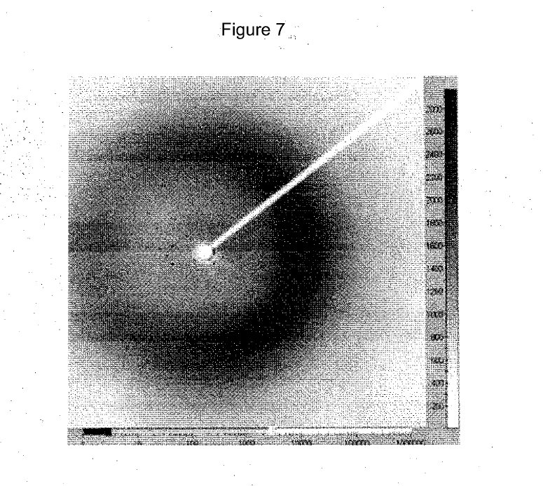

FIG. 7. The X-ray Diffraction of rsIFN-co Crystal

### DETAILED DESCRIPTION OF THE INVENTION

This invention provides a set of methods for modulating protein spatial configuration. First, select the amino-acid codon for encoding the target protein according to host codon usage. Second, choose combinations which can modulate the spatial configuration and construct into different vectors which can transfect a series of hosts. Therefore, an appropriate vector with appropriate host may be chosen. Third, choose the vector promoter by monitoring a combination of base pairs after combining the code sequence of the promoter and the target protein. Finally, choose the appropriate expression host to express the target protein, refold and purify, measure the activity and spatial configuration.

This invention discovered that during the protein-constructing process, the variation of codon that encodes the amino acid of target protein, the difference of choosing vectors, the modulation of the promoter and the selection of host expression vector, even conditions of denaturation and renaturation, agents etc. are all adjustable factors for modulating the spatial configuration of target proteins. Accordingly, modulation of the spatial configuration of proteins to obtain new functions and to improve activity is the result of systematic analysis.

This invention provides a method for modulating the function of proteins without changing the primary amino acid sequence of said protein comprising steps of: a) altering the codon usage of said protein; b) expressing the protein using the altered codon to obtain purified protein; and c) comparing the expressed protein with altered codon usage to one without, wherein an increase in function or identification of new function indicates that the function of the protein has been modulated.

In an embodiment, the altered codon usage results in high expression of said protein.

This invention also provides a method for preparing protein with enhanced or new functions without changing the primary amino acid sequence of said protein comprising steps of: a) altering the codon usage of said protein; b) expressing the protein using the altered codon to obtain purified protein; and c) comparing the expressed protein with altered codon usage to one without, wherein an increase in function or identification of new function indicates that a protein with enhanced and new function has been prepared.

In an embodiment, the altered codon usage results in high expression of said protein. This invention also provides the protein prepared by the above method. In an embodiment, the protein has unique secondary or tertiary structure.

This invention further provides a synthetic gene with altered codon, which, when expressed, produces enhanced or new functions. In an embodiment, the invention provides a vector comprising the gene. In a further embodiment, this invention provides an expression system comprising the gene. In yet a further embodiment, this invention provides a host cell comprising the gene.

This invention also provides a process for production of a protein of enhanced function or new function comprising introducing an artificial gene with selected codon preference into an appropriate host, culturing said introduced host under appropriate conditions for the expression of said protein, and harvesting the expressed protein.

This invention provides the above process, wherein the artificial gene is operatively linked to a vector. In an embodiment, the process comprises extraction of the protein from fermentation broth, or collection of the inclusion body, and denaturation and renaturation of the harvested protein.

This invention also provides the protein produced by any of the above processes.

This invention provides a composition comprising any of the above proteins and a suitable carrier. This invention further provides a pharmaceutical composition comprising any of the above produced proteins and a pharmaceutically acceptable carrier.

One significance of this invention is that it modulates the spatial configuration of protein during the process of translating genes with therapeutic effects into proteins which possess functions originating from the genes, or functions not seen in proteins produced using traditional techniques, or even with improved activity compared with those existing proteins.

Taking the interferon as an example, construct the gene of human IFN-α into reverse transcriptive expression vector to produce PDOR-INF-α expression vector, then transfect 2.2.15 cell. HBsAg and HBeAg in the culturing supernatant of cell is measured. The results indicate that the suppression rate of rSIFN-co to HBsAg was 62% and 67.7% to HBeAg, but the recombinant interferon protein produced by gene recombination techniques do not have the effect in vitro. In addition, the experiment of constructing the human INF-α2 expression vector using the reverse transcriptive viral vector and transfecting it into HIV cell strain-A3.01 proved that IFN-α2 can completely restrain the replication and transcript of HIV-DNA. However, the effect of interferon is limited in the treatment of HIV disease.

This invention will be better understood from the examples which follow. However, one skilled in the art will readily appreciate that the specific methods and results discussed are merely illustrative of the invention as described more fully in the claims which follow thereafter.

### Example 1

### Conformation Reconstruction of IFN-CONL

rSIFN-co is a new interferon molecule constructed according to conservative amino acids in human IFN-α subtype with genetic engineering methods. The interferon has been described in U.S. Pat. Nos. 4,695,263 and 4,897,471, and has been proven in literature and patents to have broad-spectrum interferon activity with strong antiviral, anti-tumor and natural cell-killing effects.

The DNA coding sequence was redesigned according to E. Coli. codon usage by first constructing an insert into pHY-vector, mediating down-stream expression with P_{BAD} promoter, then choosing E. Coli. as host. The high-purity products are gained by denaturation with 6 mol/L guanidine hydrochloride→renatured with 4 mol/L arginine→purified with Cu²⁺-chelating affinity chromatography after POROS HS/M cation exchange chromatography.

The comparison test of duplicates of hepatitis B virus DNA and secretion of HBsAg and HBeAg inhibition between rSIFN-co and IFN-con₁ proved that rSIFN-co has the effect of inhibiting the secretion of HBsAg and HBeAg which is not possessed by IFN-conl. In another test, the HBV core/pregenomic (C/P) promoter and associate cis-acting element were placed upstream of luciferase-encoding plasmid. This reporter construct was transfected into HpeG2 cells. The cells were treated with different interferons and luciferase reporter gene expression was measured. Results show that rSIFN-co can suppress 68% of luciferase reporter gene expression; whereas IFN-conl and IFN-α2b only suppress 35% and 27% of it. Therefore, the suppression effect of rSIFN-co on HBcAg has been obviously improved.

Meanwhile, circular dichroism spectrum also proved there are differences in the secondary structure of rSIFN-co by comparison with IFN-con1.

The following are those comparison experiments in detail:

### 1) Comparison of Circular Dichroism Spectrum

Address: The Center of Analysis and Test in Sichuan University

Apparatus: J-500C Circular Dichroism equipment (spectrum range: 250-190 nm/sensibility: 2 m°/cm/light path: 0.2 cm. (See FIG. 1 and FIG. 2.)

2) rSIFN-co Inhibits HBV-DNA Duplication and Secretion of HBsAg and HBeAg.

### Materials

Solvent and Dispensing Method: Add 1 ml saline into each vial, dissolve, and mix with MEM culture medium at different concentrations. Mix on the spot.

Control drugs: IFN-α2b (Intron A) as lyophilized powder, purchased from Schering Plough. 3×10⁶ U each, mix to 3×10⁶ IU/ml with culture medium; INFERGEN (liquid solution), purchased from Amgen, 9 μg, 0.3 ml each, equal to 9×10⁶ IU, and mix with 9×10⁶ IU/ml culture medium preserve at 4° C.; 2.2.15 cell: 2.2.15 cell line of hepatoma (Hep G2) cloned and transfected by HBV DNA, constructed by Mount Sinai Medical Center.

Reagent: MEM powder, Gibco American Ltd. cattle fetal blood serum, HycloneLab American Ltd. G-418 (Geneticin); MEM dispensing, Gibco American Ltd.; L-Glutamyl, imported and packaged by JING KE Chemical Ltd.; HBsAg and HBeAg solid-phase radioimmunoassay box, Northward Reagent Institute of Chinese Isotope Ltd.; Biograncetina, Northern China Medicine; and Lipofectin, Gibco American Ltd.

Experimental goods and equipment: culture bottle, Denmark Tunclon™; 24-well and 96-well culture board, Corning American Ltd.; Carbon Dioxide hatching box, Shel-Lab American Ltd.; MEM culture medium 100 ml: 10% cattle fetal blood serum, 0.03% Glutamine, G418 380 μg/ml, biograncetina 50 U/ml.

Method:

2.2.15 cell culture: Add 0.25% pancreatic enzyme into culture box with full of 2.2.15 cell. Digest at 37° C. for 3 minutes and add culture medium to stop digestion and disperse the cells. Reproduce with a ratio of 1:3. They will reach full growth in 10 days.

Toxicity test: Set groups of different concentrations and a control group in which cells are not acted on with medicine. Digest cells, and dispense to a 100,000 cell/ml solution. Inoculate to 96-well culture board, 200 μl per well. Culture at 37° C. for 24 h with 5% CO₂. Test when simple cell layer grows.

Dispense rSIFN-co to 1.8×10⁷ IU/ml solution then prepare a series of solutions diluted at two-fold gradients. Add into 96-well culture board, 3 wells per concentration. Change the solution every 4 days. Test cytopathic effect by microscope after 8 days. Fully destroy as 4, 75% as 3, 50% as 2, 25% as 1, zero as 0. Calculate average cell lesions and inhibition rates at different concentrations. Calculate TC50 and TC0 according to the Reed Muench method.

$$TC 50 = Antilog ( B + \frac{50 - B}{A - B} \times C )$$

A=log>50% medicine concentration; B=log<50% medicine concentration; C=log dilution power

Inhibition test for HBeAg and HBsAg: Separate into positive and negative HBeAg and HBsAg contrast groups, cell contrast groups and medicine concentration groups. Inoculate 700,000 cells/ml of 2.2.15 cell into 6-well culture board, 3 ml per well, culture at 37° C. for 24 h with 5% CO₂, then prepare 5 gradiently diluted solutions with 3-fold as the grade (Prepare 5 solutions, each with a different protein concentration. The concentration of Solution 2 is 3 times lower than that of Solution 1, the concentration of Solution 3 is 3 times lower than that of Solution 2, etc.) 4.5×10⁶ IU/ml, 1.5×10⁶ IU/ml, 0.5×10⁶ IU/ml, 0.17×10⁶1 U/ml, and 0.056×10⁶1 U/ml, 1 well per concentration, culture at 37° C. for 24 h with 5% CO₂. Change solutions every 4 days using the same solution. Collect all culture medium on the 8^{th} day. Preserve at −20° C. Repeat test 3 times to estimate HBsAg and HBeAg with solid-phase radioimmunoassay box (Northward Reagent Institute of Chinese Isotope Ltd.). Estimate cpm value of each well with a γ-accounting machine.

Effects calculation: Calculate cpm mean value of contrast groups and different-concentration groups and their standard deviation, P/N value such as inhibition rate, IC50 and SI.

1) Antigen inhibition rate

$$( \% ) = \frac{A - B}{A} \times 100$$

A=cpm of control group; B=cpm of test group;

2) Counting the half-efficiency concentration of the medicine

Antigen inhibition

$$TC 50 = Antilog ( B + \frac{50 - B}{A - B} \times C )$$

A=log>50% medicine concentration; B=log<50% medicine concentration; C=log dilution power

3) SI of interspace-conformation changed rSIFN-co effect on HBsAg and HBeAg in 2.2.15 cell culture:

$$SI = \frac{TC 50}{IC 50}$$

4) Estimate the differences in cpm of each dilution degree from the control group using student t test

Southern blot: (1) HBV-DNA extract in 2.2.15 cell: Culture cell 8 days. Exsuction culture medium (Separate cells from culture medium by means of draining the culture medium). Add lysis buffer to break cells, then extract 2 times with a mixture of phenol, chloroform and isoamyl alcohol (1:1:1), 10,000 g centrifuge. Collect the supernatant adding anhydrous alcohol to deposit nucleic acid. Vacuum draw, re-dissolve into 20 μlTE buffer. (2) Electrophoresis: Add 6×DNA loading buffer, electrophoresis on 1.5% agarose gel, IV/cm, at fixed pressure for 14-18 h. (3) Denaturation and hybridization: respectively dip gel into HCl, denaturaion buffer and neutralization buffer. (4) Transmembrane: Make an orderly transfer of DNA to Hybond-N membrane. Bake, hybridize and expose with dot blot hybridization. Scan and analyze relative density with gel-pro software. Calculate inhibition rate and IC50.

### Results

Results from Tables 1, 2 and 3 show: After maximum innocuous concentration exponent culturing for 8 days with 2.2.15 cell, the maxima is 9.0±0×10⁶ IU/ml average inhibition rate of maximum innocuous concentration rSIFN-co to HBeAg is 46.0±5.25% (P<O. 001), IC50 is 4.54±1.32×10⁶ IU/ml, SI is 3.96; rate to HBsAg is 44.8±6.6%, IC50 is 6.49±0.42×10⁶ IU/ml, SI is 2.77. This shows that rSIFN-co can significantly inhibit the activity of HBeAg and HBsAg, but that the IFN of the contrast group and INFERGEN cannot. It has also been proven in clinic that rSIFN-co can decrease HBeAg and HBsAg or return them to normal levels.

TABLE 1
Results of inhibition rate of rSIFN-co to HBsAg and HBeAg

| | | | | Inhibition rate | | | Average | | | Accumulated |
|---|---|---|---|---|---|---|---|---|---|---|
| Concentration | First | Second | Third | First | Second | Third | inhibition | Accum- | 1- | inhibition |
| (×10⁴ IU/ml) | well | well | well | well | well | well | rate | ulation | Accumulation | rate |

First batch: (rSIFN-co)
Inhibition effect to HBeAg

| | | | | | | | | | | |
|---|---|---|---|---|---|---|---|---|---|---|
| 900 | 9026 | 8976 | 10476 | 0.436227 | 0.43935 | 0.345659 | 0.407079 | 0.945909 | 0.592921 | 0.614693546 |
| 300 | 9616 | 12082 | 10098 | 0.3993754 | 0.245347 | 0.369269 | 0.337997 | 0.5388299 | 1.254924 | 0.300392321 |
| 100 | 9822 | 16002 | 12800 | 0.386508 | 0.0005 | 0.2005 | 0.195836 | 0.200833 | 2.059088 | 0.08867188 |
| 33.33333 | 15770 | 19306 | 16824 | 0.014991 | 0 | 0 | 0.004997 | 0.0049969 | 3.054091 | 0.001633453 |
| 11.11111 | 19172 | 22270 | 18934 | 0 | 0 | 0 | 0 | 0 | 4.054091 | 0 |
| Control | Cell | 16010 | | Blank | 0 | | Dilution | 3 | IC50 | 602.74446016 |

Inhibition effect to HBsAg

| | | | | | | | | | | |
|---|---|---|---|---|---|---|---|---|---|---|
| 900 | 7706 | 7240 | 7114 | 0.342155 | 0.381936 | 0.392693 | 0.372261 | 0.922258 | 0.627739 | 0.595006426 |
| 300 | 8856 | 7778 | 9476 | 0.2439816 | 0.336008 | 0.191053 | 0.257014 | 0.5499972 | 1.370724 | 0.286349225 |
| 100 | 10818 | 10720 | 10330 | 0.07649 | 0.084856 | 0.118149 | 0.093165 | 0.292983 | 2.27756 | 0.113977019 |
| 33.33333 | 10744 | 11114 | 10570 | 0.082807 | 0.051221 | 0.097661 | 0.07723 | 0.1998179 | 3.20033 | 0.058767408 |
| 11.11111 | 10672 | 9352 | 10810 | 0.088953 | 0.201639 | 0.077173 | 0.122588 | 0.122588 | 4.077742 | 0.02918541 |
| Control | Cell | 11714 | | Blank | 0 | | Dilution | 3 | IC50 | 641.7736749 |

Second batch: (rSIFN-co)
Inhibition effect to HBeAg

| | | | | | | | | | | |
|---|---|---|---|---|---|---|---|---|---|---|
| 900 | 7818 | 8516 | 9350 | 0.554378 | 0.514592 | 0.467054 | 0.512008 | 1.371181 | 0.487992 | 0.737521972 |
| 300 | 10344 | 10628 | 9160 | 0.4103967 | 0.394209 | 0.477884 | 0.427497 | 0.8591731 | 1.060496 | 0.447563245 |
| 100 | 12296 | 14228 | 13262 | 0.299134 | 0.18901 | 0.244072 | 0.244072 | 0.4316522 | 1.816423 | 0.19201839 |
| 33.33333 | 15364 | 17414 | 16188 | 0.124259 | 0.00741 | 0.77291 | 0.069653 | 0.1876045 | 2.74677 | 0.063933386 |
| 11.11111 | 17386 | 13632 | 15406 | 0.009006 | 0.222982 | 0.121865 | 0.117951 | 0.117951 | 3.628819 | 0.03148073 |
| Control | Cell | 16962 | | Blank | 0 | | Dilution | 3 | IC50 | 365.9357846 |

Inhibition effect to HBsAg

| | | | | | | | | | | |
|---|---|---|---|---|---|---|---|---|---|---|
| 900 | 5784 | 6198 | 5792 | 0.498265 | 0.462353 | 0.497571 | 0.486063 | 0.893477 | 0.513937 | 0.634835847 |
| 300 | 7150 | 8534 | 8318 | 0.379771 | 0.259715 | 0.278452 | 0.30598 | 0.4074138 | 1.207957 | 0.252210647 |
| 100 | 9830 | 11212 | 10210 | 0.147294 | 0.027412 | 0.11433 | 0.096345 | 0.101434 | 2.111612 | 0.04583464 |
| 33.33333 | 13942 | 12368 | 13478 | 0 | 0 | 0 | 0 | 0.0050891 | 3.111612 | 0.001632835 |
| 11.11111 | 12418 | 11634 | 11352 | 0 | 0 | 0.015267 | 0.005089 | 0.005089 | 4.106523 | 0.001237728 |
| Control | Cell | | | Blank | 0 | | Dilution | 3 | IC50 | 611.0919568 |

Third batch: (rSIFN-co)
Inhibition effect to HBeAg

| | | | | | | | | | | |
|---|---|---|---|---|---|---|---|---|---|---|
| 900 | 9702 | 9614 | 8110 | 0.428016 | 0.433204 | 0.521872 | 0.461031 | 1.316983 | 0.538969 | 0.709599543 |
| 300 | 8914 | 10032 | 8870 | 0.4744723 | 0.40856 | 0.477066 | 0.453366 | 0.8559525 | 1.085603 | 0.440859127 |
| 100 | 16312 | 12688 | 13934 | 0.038321 | 0.251975 | 0.178517 | 0.156271 | 0.402586 | 1.929332 | 0.172641621 |
| 33.33333 | 15080 | 12814 | 13288 | 0.110954 | 0.244547 | 0.216602 | 0.190701 | 0.2463153 | 2.738631 | 0.082519158 |
| 11.11111 | 21928 | 15366 | 15728 | 0 | 0.094093 | 0.072751 | 0.0055615 | 0.055615 | 3.683017 | 0.014875633 |
| Control | Cell | 17544 | | Blank | 0 | | Dilution | 3 | IC50 | 382.0496935 |

Inhibition effect to HBsAg

| | | | | | | | | | | |
|---|---|---|---|---|---|---|---|---|---|---|
| 900 | 5616 | 6228 | 5346 | 0.496864 | 0.442035 | 0.521054 | 0.486651 | 0.763125 | 0.513349 | 0.597838293 |
| 300 | 8542 | 8590 | 7096 | 0.234725 | 0.230425 | 0.364272 | 0.276474 | 0.2764738 | 1.236875 | 0.182690031 |
| 100 | 11420 | 11360 | 11394 | 0 | 0 | 0 | 0 | 0 | 2.236875 | 0 |
| 33.33333 | 12656 | 11582 | 13110 | 0 | 0 | 0 | 0 | 0 | | 0 |
| 11.11111 | 13142 | 12336 | 13342 | 0 | 0 | 0 | 0 | 0 | 4.236875 | 0 |
| Control | Cell | 11528 | | Blank | 0 | | Dilution | 3 | IC50 | 694.7027149 |

HBeAg: Average IC50: 450.2434 SD: 132.315479
HBsAg: Average IC50: 649.1894 SD: 42.29580

TABLE 2
Results of inhibition rate of Intron A(IFN-α2b) to HBsAg and HBeAg

| | | | | Inhibition rate | | | Average | | | Accumulated |
|---|---|---|---|---|---|---|---|---|---|---|
| Concentration | First | Second | Third | First | Second | Third | inhibition | Accum- | 1- | inhibition |
| (×10⁴ IU/ml) | well | well | well | well | well | well | rate | ulation | Accumulation | rate |

Inhibition effect to HBeAg

| | | | | | | | | | | |
|---|---|---|---|---|---|---|---|---|---|---|
| 300 | 14918 | 11724 | 9950 | 0 | 0.029711 | 0.176529 | 0.068747 | 0.068747 | 0.931253 | 0.068746724 |
| 100 | 14868 | 16890 | 15182 | 0 | 0 | 0 | 0 | 0 | 1.931253 | 0 |
| 33.33333 | 16760 | 21716 | 16400 | 0 | 0 | 0 | 0 | 0 | 2.931253 | 0 |
| 11.11111 | 20854 | 15042 | 16168 | 0 | 0 | 0 | 0 | 0 | 3.931253 | 0 |
| 3.703704 | 12083 | 12083 | 12083 | 0 | 0 | 0 | 0 | 0 | 4.931253 | 0 |
| Control | Cell | 17544 | | Blank | 0 | | Dilution | 3 | IC50 | FALSE |

Inhibition effect to HBsAg

| | | | | | | | | | | |
|---|---|---|---|---|---|---|---|---|---|---|
| 300 | 9226 | 8196 | 9658 | 0.152489 | 0.247106 | 0.521054 | 0.1708 | 0.189295 | 0.8292 | 0.185857736 |
| 100 | 10946 | 10340 | 10828 | 0 | 0.050156 | 0.364272 | 0.018495 | 0.0184947 | 1.810705 | 0.010110817 |
| 33.33333 | 12250 | 12980 | 13934 | 0 | 0 | 0 | 0 | 0 | 2.810705 | 0 |
| 11.11111 | 12634 | 12342 | 12000 | 0 | 0 | 0 | 0 | 0 | 3.810705 | 0 |
| 3.703704 | 10886 | 10886 | 10886 | 0 | 0 | 0 | 0 | 0 | 4.810705 | 0 |
| Control | Cell | 10886 | | Blank | 0 | | Dilution | 3 | IC50 | FALSE |

TABLE 3
Results of inhibition rate of Infergen to HBsAg and HBeAg

| | | | | Inhibition rate | | | Average | | | Accumulated |
|---|---|---|---|---|---|---|---|---|---|---|
| Concentration | First | Second | Third | First | Second | Third | inhibition | Accum- | 1- | inhibition |
| (×10⁴ IU/ml) | well | well | well | well | well | well | rate | ulation | Accumulation | rate |

First batch: (Infergen)
Inhibition effect to HBeAg

| | | | | | | | | | | |
|---|---|---|---|---|---|---|---|---|---|---|
| 900 | 14172 | 12156 | 17306 | 0.091655 | 0.220869 | 0 | 0.104175 | 0.306157 | 0.895825 | 0.254710274 |
| 300 | 13390 | 12288 | 16252 | 0.1417767 | 0.212409 | 0 | 0.118062 | 0.2019827 | 1.777764 | 0.102024519 |
| 100 | 14364 | 18834 | 14194 | 0.079349 | 0 | 0.090245 | 0.056531 | 0.083921 | 2.721232 | 0.029916678 |
| 33.33333 | 15722 | 16034 | 16340 | 0 | 0 | 0 | 0 | 0.0273897 | 3.721232 | 0.007306592 |
| 11.11111 | 17504 | 17652 | 14320 | 0 | 0 | 0.082169 | 0.02739 | 0.02739 | 4.693843 | 0.005801377 |
| Control | Cell | 15602 | | Blank | 0 | | Dilution | 3 | IC50 | FALSE |

Inhibition effect to HBsAg

| | | | | | | | | | | |
|---|---|---|---|---|---|---|---|---|---|---|
| 900 | 12080 | 11692 | 12234 | 0 | 0.01275 | 0 | 0.00425 | 0.025163 | 0.99575 | 0.024647111 |
| 300 | 12840 | 11484 | 12350 | 0 | 0.030313 | 0 | 0.010104 | 0.0209125 | 1.985646 | 0.010422073 |
| 100 | 12894 | 14696 | 15086 | 0 | 0 | 0 | 0 | 0.010808 | 2.985646 | 0.003606955 |
| 33.33333 | 15032 | 12928 | 13020 | 0 | 0 | 0 | 0 | 0.0108081 | 3.985646 | 0.002704416 |
| 11.11111 | 11794 | 11984 | 11508 | 0.004137 | 0 | 0.028287 | 0.010808 | 0.010808 | 4.974837 | 0.002167838 |
| Control | Cell | 11843 | | Blank | 0 | | Dilution | 3 | IC50 | FALSE |

Second batch: (Infergen)
Inhibition effect to HBeAg

| | | | | | | | | | | |
|---|---|---|---|---|---|---|---|---|---|---|
| 900 | 6278 | 6376 | 6408 | 0.200051 | 0.187564 | 0.183486 | 0.190367 | 0.274635 | 0.809633 | 0.253290505 |
| 300 | 7692 | 9092 | 6394 | 0.0198777 | 0 | 0.18527 | 0.068383 | 0.0842678 | 1.74125 | 0.046161005 |
| 100 | 8960 | 7474 | 8190 | 0 | 0.047655 | 0 | 0.015885 | 0.015885 | 2.725365 | 0.005794856 |
| 33.33333 | 8530 | 8144 | 9682 | 0 | 0 | 0 | 0 | 0 | 3.725365 | 0 |
| 11.11111 | 7848 | 7848 | 7848 | 0 | 0 | 0 | 0 | 0 | 4.725365 | 0 |
| Control | Cell | 7848 | | Blank | 0 | | Dilution | 3 | IC50 | FALSE |

Inhibition effect to HBsAg

| | | | | | | | | | | |
|---|---|---|---|---|---|---|---|---|---|---|
| 900 | 12364 | 12268 | 12274 | 0.036171 | 0.043655 | 0.043187 | 0.041004 | 0.140162 | 0.958996 | 0.12751773 |
| 300 | 11590 | 12708 | 13716 | 0.0965076 | 0.009355 | 0 | 0.035287 | 0.0991581 | 1.923709 | 0.0490186 |
| 100 | 12448 | 13468 | 13982 | 0.029623 | 0 | 0 | 0.009874 | 0.063871 | 2.913834 | 0.02144964 |
| 33.33333 | 12616 | 11346 | 12444 | 0.016526 | 0.115529 | 0.029935 | 0.053996 | 0.0539965 | 3.859838 | 0.013796309 |
| 11.11111 | 12828 | 12828 | 12828 | 0 | 0 | 0 | 0 | 0 | 4.859838 | 0 |
| Control | Cell | 12828 | | Blank | 0 | | Dilution | 3 | IC50 | FALSE |

Third batch: (Infergen)
Inhibition effect to HBeAg

| | | | | | | | | | | |
|---|---|---|---|---|---|---|---|---|---|---|
| 900 | 7240 | 6642 | 6158 | 0.064599 | 0.14186 | 0.204393 | 0.136951 | 0.217399 | 0.863049 | 0.201211735 |
| 300 | 11072 | 8786 | 6902 | 0 | 0 | 0.108269 | 0.03609 | 0.0804479 | 1.82696 | 0.042176564 |
| 100 | 7016 | 9726 | 7552 | 0.09354 | 0 | 0.024289 | 0.039276 | 0.044358 | 2.787683 | 0.015663017 |
| 33.33333 | 7622 | 8866 | 8676 | 0.015245 | 0 | 0 | 0.005082 | 0.0050818 | 3.782601 | 0.001341671 |
| 11.11111 | 7740 | 7740 | 7740 | 0 | 0 | 0 | 0 | 0 | 4.782601 | 0 |
| Control | Cell | 7740 | | Blank | 0 | | Dilution | 3 | IC50 | FALSE |

Inhibition effect to HBsAg

| | | | | | | | | | | |
|---|---|---|---|---|---|---|---|---|---|---|
| 900 | 11048 | 11856 | 11902 | 0.04775 | 0 | 0 | 0.015917 | 0.015917 | 0.984083 | 0.015916796 |
| 300 | 13454 | 12896 | 11798 | 0 | 0 | 0 | 0 | 0 | 1.984083 | 0 |
| 100 | 12846 | 13160 | 12546 | 0 | 0 | 0 | 0 | 0 | 2.984083 | 0 |
| 33.33333 | 12680 | 12458 | 12360 | 0 | 0 | 0 | 0 | 0 | 3.984083 | 0 |
| 11.11111 | 11602 | 11602 | 11602 | 0 | 0 | 0 | 0 | 0 | 4.984083 | 0 |
| Control | Cell | 11602 | | Blank | 0 | | Dilution | 3 | IC50 | FALSE |

HBeAg: Average IC50: 0 SD: 0
HBsAg: Average IC50: 0 SD: 0

### Example 2

### Comparison of Inhibitory Effects of Different Interferons On HBV Gene Expression

Hepatitis B virus (HBV) DNA contains consensus elements for transactivating proteins whose binding activity is regulated by interferons. Treatment of HBV-infected hepatocytes with interferons leads to inhibition of HBV gene expression. The aim of the present study was to characterize the effects of different interferons on HBV regulated transcription. Using transient transfection of human hepatoma cells with reporter plasmids containing the firefly luciferase gene under the control of HBV-Enhancer (EnH) I, Enh II and core promoter, Applicant studied the biological activities of three different interferons on transcription.

### Materials and Methods

1. Interferons: IFN-con1 (Infergen®), IFN-Hui-Yang (γSIFN-co) and IFN-beta 1b

2. Reporter plasmid: The DNA fragments containing HBV-Enhancer (EnH) I, Enh II and core promoter were prepared using PCR and blunt-end cloned into the Smal I site of the promoter- and enhancer-less firefly luciferase reporter plasmid pGL3-Basic (Promega, Wis., USA). The resulting reporter plasmid was named as pGL3-HBV-Luc.

3. Cell Culture and DNA transfection: HepG2 cells were cultured in DMEM medium supplemented with 10% FBS and 100 U/ml penicillin and 100 ug/ml streptomycin. The cells were kept in 30° C., 5% CO2 incubator. The cells were transfected with pGL3-HBV-Luc reporter plasmid using Boehringer's Lipofectin transfection kit. After 18 hours, the medium containing transfection reagents was removed and fresh medium was added with or without interferons. The cells were kept in culture for another 48 hours.

4. Luciferase Assay: Forty-eight hours after the addition of interferon, the cells were harvested and cell lysis were prepared. The protein concentration of cell lysates were measured using Bio-Rad Protein Assay kit. The luciferase activity was measured using Promega's Luciferase Reporter Assay Systems according to the instructions of manufacturer.

### Results

### Expression of Luciferase Activity in Different Interferon-Treated Cell Lysates

| No treatment | IFN-con1 | IFN-Hui-Yang | IFN-beta 1b |
|---|---|---|---|
| 100 | 48 + 8 | 29 + 6 | 64 + 10 |

This result shows that γSIFN-co inhibits most effectively on the expression of HBV gene expression.

### Example 3

### Side Effects and Changes in Body Temperature when Using γSIFN-co

There are usually more side effects to using interferon. The side effects include: nausea, muscle soreness, loss of appetite, hair loss, hypoleucocytosis (hypoleukmia; hypoleukocytosis; hypoleukia), and decrease in blood platelets, etc.

### Method

Sample patients are divided into two groups. 11 patients in Group A were injected with 9 μg Infergen®. 10 patients in Group B were injected with 9 μg γSIFN-co. Both groups were monitored for 48 hours after injections. First monitoring was recorded 1 hour after injection, after that, records were taken every 2 hours.

Table 4 is the comparison of side effects between patients being injected with 9 μg of Infergen® and 9 μg of γSIFN-co.

TABLE 4
Side Effects

| | | γSIFN-co | Infergen ® |
|---|---|---|---|
| | | 9 μg | 9 μg |
| | | Person: n = 10 | Person: n = 11 |
| Body Systems | Reactions | Headcount | Headcount |
| In General | Feebleness | 3 | 3 |
| | Sole heat | 1 | |
| | Frigolability | 3 | 4 |
| | Decrease in | | 3 |
| | leg strength | | |
| | Mild lumbago | 2 | 1 |
| | Body soreness | 4 | 5 |
| Central Nervous | Headache | 3 | 6 |
| System/ | Dizziness | 2 | 11 |
| Peripheral | Drowsiness | | 3 |
| Nervous System | | | |
| Gastroenterostomy | Apoclesis | 1 | |
| | Celiodynia | 1 | |
| | Diarrhea | 1 | |
| Musculoskeletal | Myalgia | 1 | 2 |
| system | | | |
| | Arthralgia | 2 | |
| Respiratory | Stuffy nose | 1 | |
| system | | | |
| Paropsia | Swollen eyes | | 1 |

### Results

For those patients who were injected with γSIFN-co, the side effects were minor. They had some common symptoms similar to flu, such as: headache, feebleness, frigolability, muscle soreness, hidrosis, and arthralgia (arthrodynia; arthronalgia). The side effects of those patients whom were injected with Infergen® were worse than those were injected with γSIFN-co.

From FIGS. 4A-1, 4A-2, 4B-1, and 4B-2, it was obvious that the body temperatures of sample patients in Group A were higher than the patients in Group B. It also reflected that the endurance of γSIFN-co was much better than Infergen®.

### Example 4

### Crystal Growth of γSIFN-co and Test of Crystallography Parameter

Crystal of γSIFN-co. Two types of crystal were found after systematic trial and experiment. (See FIGS. 5-7)

### 1. Crystal Growth

Dissolve γSIFN-co protein with pure water (H2O) to 3 mg/ml in density. Search crystallization by using Hampton Research Crystal Screen I and II which was made by Hampton Company. By using prop Suspension Diffusion Method, liquid 500 μl, drop 1 μl protein+1 μl liquid, in 293K temperature. First 2 different types of small crystals were found as listed in Table 5.

TABLE 5
Screen of γSIFN-co Crystallin

| | Condition | |
|---|---|---|
| | I | II |
| Diluent | 0.1M Tris-HCl | 0.1M HEPES |
| | PH = 8.75 | PH = 7.13 |
| Precipitant | 17.5% (w/v) PEG550 MME | 10% (w/v)PEG6K |
| Additives | 0.1M NaCl | 3% (v/v)MPD |
| Temperature | 293 K | 293 K |
| Crystal Size (mm) | 0.2 × 0.2 × 0.1 | 0.6 × 0.02 × 0.02 |
| Crystallogram | FIG. 5 | FIG. 6 |

### 2. Data Collection and Processing

Crystal I was used to collect X-Ray diffraction data and preliminary analysis of crystallography. Testing of parameters was also completed. The diffraction data was collected under room temperature. Crystal I (Condition I) was inserted into a thin siliconized wall tube. By using BrukerAXS Smart CCD detector, light source CuKα (λ=1.5418 Å) generated by Nonius FR591 X-ray generator. Light power 2000 KW (40 kv×50 mA), wave length 1.00 Å, under explosion 60 second, Δφ=2°, the distance between crystal and detector was 50 mm. Data was processed using Proteum Procedure Package by Bruker Company. For crystal diffraction pattern (partially), see FIG. 7. See Table 6 for process results.

TABLE 6
Results of Crystallography Parameters

| Parameters | |
|---|---|
| a (Å) | 82.67 |
| b (Å) | 108.04 |
| c (Å) | 135.01 |
| α (°) | 90.00 |
| β (°) | 90.00 |
| γ (°) | 98.35 |

Space Group P2 or P2₁
Sharpness of separation 5 Å
Asymmetric molecule # 10
Dissolution 57.6%

In addition, there was no crystal growth of γSIFN-co based on previous publications. The closest result to the γSIFN-co was huIFN-a2b but the screen was very complicated. After seeding 3 times, crystal grew to 0.5×0.5×0.3 mm, sharpness of separation was 2.9 Å, space group was P2₁. The crystals were also big, asymmetric molecule number was 6, and dissolution was about 60%.

1. A method for modulating the function of proteins without changing the primary amino acid sequence of said protein comprising steps of:
a) altering the codon usage of said protein;
b) expressing the protein using the altered codon to obtain purified protein; and
c) comparing the expressed protein with altered codon usage to one without, wherein an increase in function or identification of new function indicates that the function of the protein has been modulated.

2. The method of claim 1, wherein the altered codon usage results in high expression of said protein.

3. (canceled)

4. The method of claim 1, wherein the altered codon usage results in high expression of said protein.

5. An isolated protein prepared by the method of claim 1.

6. The protein of claim 5 with unique secondary or tertiary structure.

7. A synthetic gene with altered codon which, when expressed, produces enhanced or new functions.

8. A vector comprising the gene of claim 7.

9. An expression system comprising the gene of claim 7.

10. A host cell comprising the gene of claim 7.

11. A process for production of a protein of enhanced function or new function comprising introducing an artificial gene with selected codon preference into an appropriate host, culturing said introduced host under appropriate conditions for the expression of said protein, and harvesting the expressed protein.

12. The process of claim 11, wherein the artificial gene is operatively linked to a vector.

13. The process of claim 11, comprising extraction of the protein from fermentation broth, or collection of the inclusion body, and denaturation and renaturation of the harvested protein.

14. An isolated protein produced by the process of claim 11.

15. A composition comprising the protein of claim 5 and a suitable carrier.

16. A pharmaceutical composition comprising the produced protein of claim 5 and a pharmaceutically acceptable carrier.
